(19) United States
(12) Patent Application Publication
(10) Pub. No.: US 2012/0095524 A1
(43) Pub. Date: Apr. 19, 2012

(54) METHOD OF COUNTERACTING SEIZURES

(75) Inventors: Timothy Scott Nelson, Victoria (AU); Mark James Cook, Victoria (AU); Anthony Neville Burkitt, Victoria (AU)

(73) Assignee: The Bionics Institute of Australia, Victoria (AU)

(21) Appl. No.: 13/381,586

(22) PCT Filed: Jun. 29, 2010
(86) PCT No.: PCT/AU2010/000816

§ 371 Date: Dec. 29, 2011

(30) Foreign Application Priority Data
Jun. 30, 2009 (AU) 2009903031

(51) Int. Cl. A61N 1/36

(52) U.S. Cl. 607/45

(57) ABSTRACT

The present invention provides a method for counteracting seizure events in a mammalian brain, the method comprising applying an electrical stimulus to the brain, the electrical stimulus being pulsatile and comprising pulses forming a pulse train. In order to effectively “desynchronize” the neural activity patterns in the brain, the pulse train can be at a frequency greater than substantially 300 Hz and at a duty cycle greater than substantially 20%, the pulse train can have an inconstant inter pulse interval such that the pulse rate is not constant throughout the pulse train, and the pulses can have a pulse width greater than substantially 300 μsec. Apparatus for carrying out the method is also described.

### CROSS-REFERENCE TO RELATED APPLICATIONS

The present application claims priority from Australian Provisional Patent Application No 2009903031 filed on 30 Jun. 2009, the content of which is incorporated herein by reference.

### FIELD OF INVENTION

The present invention relates to use of an electrical stimulus of specific characteristic, applied by electrodes placed within or near the source of seizures in the brain, to terminate or at least counteract epileptic seizure events arising from that source.

### BACKGROUND OF THE INVENTION

Epilepsy is a chronic neurological disorder characterised by recurring unprovoked seizures, which are symptoms of episodic abnormal electrical activity in the brain. Epilepsy is a group of syndromes with widely varying symptoms, but all involving abnormal neuronal activity. Epilepsy is quite common, with about 0.5-1% of the population having active epilepsy at any time.

Generalized epilepsy is produced by electrical activity that arises substantially simultaneously throughout the entire brain. In contrast partial seizures are produced, at least initially, by electrical impulses that are generated in a relatively small part of the brain, referred to as the focus.

Medicines exist that can assist in controlling epileptic seizures in some patients. For drug refractory epilepsy patients, efforts are made to locate the focus causing the seizures with a view to surgical removal of a lesion or the like at that location, if possible. However, only a small proportion of drug refractory epilepsy patients can be treated surgically. To provide another treatment option, recent work has used implanted electrical devices to apply electrical stimuli. Under a number of different stimulation approaches, the stimuli have been applied to either the vagus nerve in the neck, to the brain surface, or to the focus within the patient's brain. Responsive stimulation involves monitoring electrical activity of the brain to detect signs that a seizure is imminent or has commenced, and applying the stimulus only in response to such detection. The applied stimulus is intended to suppress the seizure.

As seen in electroencephalography (EEG), normal ongoing brain activity is mostly in the frequency range of 1 Hz (sleep) to 40 Hz (gamma range, active concentration), and is predominantly between 1 and 20 Hz. Epileptiform electrical activity can be abnormally synchronous, and one previous approach applies a stimulus configured to desynchronise such electrical activity. Another approach is to apply stimuli at a fixed rate of 130 Hz.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

the present invention utilises novel ranges of electrical stimulus parameters to “desynchronize” the neural activity patterns in the brain. In accordance with one or more aspects of the invention that will now be described, relatively high electrical pulse rates, long electrical pulse widths and high duty cycles, and/or asynchronous electrical pulses are used. The use of high electric pulse rates produces excitation in bursts at times between the lower rate epileptiform synchronous activity in the brain. The use of long electrical pulse widths and high duty cycles allows excitation to spread out in time and occur at any time during the electrical pulses, reducing the synchrony of the excitation of the population of neurons close to the site of stimulation. The use of asynchronous electrical pulses with pseudo-random interpulse intervals introduces asynchronous excitation into the neural activity. Thus the invention introduces a larger number of asynchronous pulsatile stimuli compared with conventional stimuli consisting of pulses at a fixed low rate. In addition, each of the pulses smears the resulting neural excitation over a longer time period by the use of longer pulse widths and greater duty cycles compared with the shorter pulse widths and lower duty cycles of conventional stimuli.

According to a first aspect, the present invention provides a method for counteracting seizure events in a mammalian brain, the method comprising applying an electrical stimulus to the brain, the electrical stimulus being pulsatile and comprising pulses forming a pulse train at a frequency greater than substantially 300 Hz and at a duty cycle greater than substantially 20%.

According to a second aspect, the present invention provides a device for counteracting seizure events in a mammalian brain, the device comprising:

at least one electrode configured to apply an electrical stimulus to the brain; and

a stimulus generator which is arranged to apply an electrical stimulation to the brain via the at least one electrode, the electrical stimulation being pulsatile and forming a pulse train comprising pulses at a frequency greater than substantially 300 Hz and at a duty cycle greater than substantially 20%.

The first and second aspects of the present invention thus recognise that application of a stimulus to counteract a seizure event can be efficacious when a pulse train is delivered at a frequency substantially higher than frequency ranges previously considered clinically relevant to electrical activity in the brain. In preferred embodiments of the invention, the pulse train is at a frequency greater than 320 Hz, more preferably the pulse train is at a frequency greater than 350 Hz, and even more preferably the pulse train is at a frequency greater than 370 Hz. In preferred embodiments of the invention, the pulse train is at a frequency less than 5 kHz, more preferably the pulse train is at a frequency less than 2.5 kHz, more preferably the pulse train is at a frequency less than 1 kHz, more preferably the pulse train is at a frequency less than 700 Hz, more preferably the pulse train is at a frequency less than 600 Hz, and even more preferably the pulse train is at a frequency less than 500 Hz. Most preferably, the electrical stimulation comprises a pulse train at a frequency of substantially 370 Hz.

The duty cycle of the pulse train is preferably greater than 30%, and more preferably is greater than 50%. Most preferably the duty cycle of the pulse train is substantially 75%. In preferred embodiments the pulse train comprises a series of bipolar charge balanced pulses to effect a substantially zero net charge transfer to the brain, and in such embodiments the duty cycle of the pulse train may be as high as 100%.

The pulse train may comprise a constant inter pulse interval such that the pulse rate is constant throughout the pulse train (periodic/synchronous). However, in preferred embodiments, the pulse train comprises variable pulse spacing throughout the pulse train such that the pulse rate is not constant throughout the pulse train (aperiodic/asynchronous). In such embodiments the average pulse rate constitutes the frequency which must be greater than 300 Hz in embodiments of the first and second aspects of the invention. For example the pulse rate may be pseudo-randomly varied about the average pulse rate, for example being varied with onset times (or interpulse intervals) as per a Poisson distribution.

According to a third aspect of the present invention, there is provided a method for counteracting seizure events in a mammalian brain, the method comprising applying an electrical stimulus to the brain, the electrical stimulus being pulsatile and comprising pulses forming a pulse train, wherein the pulse train has an inconstant inter pulse interval such that the pulse rate is not constant throughout the pulse train.

According to a fourth aspect of the present invention, there is provided a device for counteracting seizure events in a mammalian brain, the device comprising:

at least one electrode configured to apply an electrical stimulus to the brain; and

a stimulus generator which is arranged to apply an electrical stimulation to the brain via the at least one electrode, the electrical stimulation being pulsatile and comprising pulses forming a pulse train, wherein the pulse train has an inconstant inter pulse interval such that the pulse rate is not constant throughout the pulse train.

The method and device of the third and fourth aspects of the present invention may be configured in accordance with the method and device of the first and second aspects of the invention. The method and device of the third and fourth aspects may have any one or more of the preferable features set forth in relation to the method and device of the first and second aspects.

In any of the aspects, preferably electrical pulses whose pulse duration (pulse width) is greater than 300 μsec are delivered to the electrode(s). For example, the pulse width may be in the range of 300-1000 μsec or 300-1500 μsec.

According to a fourth aspect of the present invention, there is provided a method for counteracting seizure events in a mammalian brain, the method comprising applying an electrical stimulus to the brain, the electrical stimulus being pulsatile and comprising pulses forming a pulse train, wherein the pulse train has an inconstant inter pulse interval such that the pulse rate is not constant throughout the pulse train, and wherein the pulses having a pulse width greater than substantially 300 μsec.

According to a fifth aspect of the present invention, there is provided a device for counteracting seizure events in a mammalian brain, the device comprising:

at least one electrode configured to apply an electrical stimulus to the brain; and

a stimulus generator which is arranged to apply an electrical stimulation to the brain via the at least one electrode, the electrical stimulation being pulsatile and comprising pulses forming a pulse train, wherein the pulse train has an inconstant inter pulse interval such that the pulse rate is not constant throughout the pulse train, and wherein the pulses having a pulse width greater than substantially 300 μsec.

The method and device of the fourth and fifth aspects of the present invention may be configured in accordance with the method and device of the first and second aspects of the invention. The method and device of the fourth and fifth aspects may have any one or more of the preferable features set forth in relation to the method and device of the first and second aspects. Nonetheless, in the fourth and fifth aspects, the average pulse rate may be lower than 300 Hz. For example, the pulse rate may be as low as 50 Hz, 100 Hz or 125 Hz.

In any of the aspects of the invention, the current intensity of the electrical stimulus may be less than 500 μA, more preferably less than 400 μA or 300 μA, and most preferably less than 200 μA. Using parameters of stimulation in accordance with the various aspects of the invention, such as frequency greater than 300 Hz, pulse width greater than 300 μsec and/or the aperiodic/asynchronous pulse train, may permit a lower current intensity to be utilised to achieve seizure control. In general, the stimulation parameters are preferentially chosen to be within a 3nC/phase limit that ensures safety for underlying neural tissue.

In any of the aspects of the present invention, the electrical stimulus may be of very short duration, for example comprising fewer than 10 pulses. Alternatively, the electrical stimulus may be applied substantially continuously over an extended period. The electrical stimulus is preferably applied to the brain for between 300 ms and 3000 ms. Pulses within the pulse train may all have the same amplitude, or may differ in amplitude. Pulses within the pulse train may all have the same pulse width, or may differ in pulse width. Different sections of the pulse train may have different pulse rates or different Poisson pulse intervals.

Some embodiments of the invention may further comprise monitoring electrical activity of the brain to detect a seizure event, and applying the electrical stimulus only in response to a seizure event being detected. For example the device may comprise a signal processor configured to analyse electrical activity of the brain detected by a sensor. The at least one electrode may serve as the sensor. In such embodiments of the invention, the stimulus is preferably initiated earlier than two seconds after onset of a seizure. In embodiments of the invention, the seizure event may comprise an impending seizure or an occurring seizure. Prediction of the impending seizure or detection of occurrence of a seizure may be performed by any suitable method.

Alternative embodiments may apply the electrical stimulus substantially continuously or at regular intervals, without attempting to detect a seizure event.

In preferred embodiments of the invention, the stimulus is applied by a plurality of electrodes. A pulse train delivered by each electrode may be the same as a pulse train delivered by each other electrode, or alternatively one or more characteristics of the pulse train may differ from one electrode to the next. For example a pulse train applied to each electrode pair may have a variable pulse spacing determined by independent Poisson processes, whether at the same average pulse rate or at distinct average pulse rates.

In embodiments of the invention, the sensor of the device may comprise one or more electrodes of an electrode array of the device. The electrode(s) used to apply the stimulus may be the sensor electrode(s).

In embodiments of the invention, the stimulus may be adaptable in response to measured brain electrical activity before, during or after application of one or more preceding stimuli. Alternatively, in some embodiments the stimulus to be applied may be fixed, with brain activity detection used only for detection of a seizure event.

Where the patient in question is known to have focal seizures, the stimulus is preferably applied to or near the focus of the seizure activity within the brain.

Embodiments of the device of the present invention may comprise:

an implantable component comprising the electrode(s) and an internal antenna; and

an external component comprising a power source, the processor, and an external antenna for wireless transcutaneous communication with the internal antenna.

The internal antenna and external antenna preferably comprise inductive coils for effecting inductive transcutaneous power transmission to power the implantable component and optionally transfer data in both directions for the detection of seizures and control of stimuli.

The mammalian brain may comprise a human brain.

### BRIEF DESCRIPTION OF THE DRAWINGS

An example of the invention will now be described with reference to the accompanying drawings, in which:

FIG. 1 illustrates one stimulation parameter search space explored by the present invention;

FIG. 2 is a diagrammatical representation of experimental stimulus delivery protocol and continuous time recording of electrophysiological signals acquired from right motor cortex during a seizure terminating therapy;

FIG. 3 illustrates a pulse generator triggering signal to stimulate from four electrodes synchronously where the inter-pulse interval follows a Poisson distribution;

FIG. 4 is a plot of mean epileptic after discharge (EAD) durations with ±95% confidence intervals (CI) versus days post surgery;

FIG. 5 is a plot of the Mann-Whitney Rank Sum test significance, P, value as a function of days post surgery for a rat (K11) EAD data;

FIG. 6 illustrates a pulse generator triggering signal to stimulate from four electrodes asynchronously where each channel pulse rate follows a unique Poisson distribution;

FIG. 7 shows the duration and treatment latency and the total EAD duration for all trials, with and without aperiodic/asynchronous, 500 Hz-based, 1000 μs stimuli;

FIG. 8 shows the duration and treatment latency and the total EAD duration for all trials, with and without aperiodic/asynchronous, 1000 Hz-based, 500 μs stimuli;

FIG. 9 shows the duration and treatment latency and the total EAD duration for all trials, with and without periodic/synchronous, 125 Hz, 300 μs stimuli;

FIG. 10 shows the duration and treatment latency and the total EAD duration for all trials, with and without periodic/synchronous, 125 Hz, 1000 μs stimuli;

FIG. 11 shows the duration and treatment latency and the total EAD duration for all trials, with and without aperiodic/asynchronous, 125 Hz, 1000 μs stimuli;

FIG. 12 shows the duration and treatment latency and the total EAD duration for all trials, with and without periodic/synchronous, 500 Hz, 1000 μs stimuli;

FIG. 13 shows the duration and treatment latency and the total EAD duration for all trials, with and without aperiodic/asynchronous, 500 Hz-based, 1000 μs stimuli; and

FIG. 14 is a schematic drawing of apparatus according to one embodiment of the present invention.

### DESCRIPTION OF THE PREFERRED EMBODIMENTS

The present invention arises from consideration of parameters of biphasic rectangular electrical pulsatile stimulation for seizure abatement, such as: the duration of each phase of the pulse, the inter-phase gap, the frequency of delivery of each pulse, the amplitude of each pulse, the time at which the pulse occurs, timing relative to the seizure onset (in the case of closed loop embodiments) and the stimulus location. In exploring this range of potential stimulation parameters, it is required to stay within safety bounds for the charge density (30 μC/cm²) and total amount of charge delivered per phase (3nC), for macro and micro electrodes respectively.

We first describe a device suitable for implementing the present invention. An implantable component takes the form of a hermetically sealed, biocompatible metal (e.g. titanium), polymer or ceramic casing that could be implanted either locally (in the skull) or remotely in the chest cavity or shoulder region of patients if required. The estimated weight of such a device would be less than 100 g and could be of a similar mass to any plate of skull removed during implantation. As the precise brain region to be recorded/stimulated will change from patient to patient, the placement of the device could be standardised to ensure a routine surgical implantation procedure. Skull site may be somewhere around the temporal/occipital region where it is less likely to sustain forceful blows, with the electrode leads crossing the dura in a single location before diverging to specific brain locations. Internal electronics of the implanted component are responsible for delivery of stimuli and acquisition of neural signals only.

The implantable component uses electrode components suitable for long term monitoring of deep brain or cortical structures, or both. Depth electrodes consist of 2-3 mm platinum electrode rings, 5-10 mm apart on a biocompatible polymer probe. The present construction of cortical electrodes is 2-4 mm diameter platinum discs mounted with 10 mm separation in a biocompatible polymer sheet. Custom electrode designs could also be used to provide higher spatial resolution placement in the areas of interest where standard spacing grid arrays may not suit. For example, 32 individual electrode sites could be implanted in and on the brain, with the intention of only 16 or less of these being monitored or stimulated at any one time. Redundancy of this nature allows for temporal changes in the epilepsy and for failure in particular electrode sites. Power demands of the device will be minimised by ensuring electrode impedances are kept as low as possible, which consequently requires high input impedance on the preamplifiers.

In this embodiment a “closed loop” approach is taken whereby stimuli are only delivered to the brain tissue in response to detection of a seizure or imminent seizure. Alternative embodiments may adopt an “open loop” approach in which stimuli are delivered without determining whether or not a seizure is occurring or imminent. In the present closed loop embodiment, stimuli should be delivered only in response to predicted or detected seizures with a latency as short as possible and preferably at most being 2 seconds or less for detection. Switching times between record and stimulate modes should not be greater than 100 μs and ideally as short as possible. The combination of electrodes used for stimulation and reference sites must be configurable at the time of implantation and through the device lifetime with the possibility to dynamically alter these sites in response to ECoG cortical signals or other response parameters.

The external component is in proximity to the implanted component but is externally located. The processor of the external component is responsible for communicating with, directing and powering the internal component. Having the intelligent components outside of the body facilitates upgrades to processor or battery capacity as new technologies become available. Specific processor selection will be dependent on the algorithms requiring implementation. To allow clinicians the best possible information for device assessment the processor should be capable of recording and storing a set amount of data around recognised seizure events. This could be achieved by maintaining a rolling five minute window of data that is used within the detection algorithm, with data older than five minutes being lost. On detecting an event this data is instead stored in the onboard memory for future review. If 4 channels were to be stored with 16-bit resolution at 200 Hz sampling rate, the data rate would be 5.6 MB per hour. With 1 GB of memory available within the device, a total of approximately 175 hours of data could be stored for review. For training and refinement purposes the processor should also be able to connect to a laptop and port all incoming data in real-time to an external proprietary capture and visualising program. This connection also allows for reprogramming of the device as individual requirements change and as algorithms for seizure detection and electrical stimulation improve.

Power transfer to the internal component, and data communication between the components, is effected by magnetic induction and is used to transmit detected signals to the external component that in turn issues stimulation commands to the internal component in response to algorithm outputs. Power is also transmitted via the induction loop rendering the internal components as simple as possible, thus minimising their chance of failure and the need for subsequent surgical intervention.

Apparatus according to one embodiment of the present invention is shown schematically in FIG. 14. The apparatus includes an implantable component 1 comprising an electrode array, which is configured for insertion in and/or on a subject's brain. The electrode array is designed to both monitor for seizure events within the brain, and to apply electrical stimulation to the brain via electrodes of the array following detection of a seizure event. In this regard, the electrode array is configured to transmit signals to, and receive signals from, a control device 2. Although the control device 2 in this embodiment is intended for use external to the patient's body, it is conceived that, like the implantable device 1, the whole or parts of the control device 2 may also be implantable.

The control device 2 comprises a switch matrix 21 connected to both a stimulator 22 and an EEG acquisition device 23. The switch matrix 21 is configured, upon control by a CPU 24, to route electrical stimulation signals from the stimulator 22 to the implantable component 1, and route seizure detection data from the implantable component 1 to the EEG acquisition device 23. The control device 2 is powered by an internal battery 25. A communications interface 26 is provided to connect the control device 2 to a user interface 3, such as a personal computer, e.g., a tablet PC 3. The user interface 3 may permit the user to monitor seizure detection information and adjust stimulation parameters. Details such as the current stimulation parameters and historical stimulation data may be stored in a digital memory 27 connected to the CPU 24 and the communications interface 26.

The communication between the implantable component 1 and the control device 2, and between the control device 2 and the user interface 3, may be wireless communication, or may be via wires.

The first and second aspects of the present invention recognise that seizures can be terminated by a sequence of electrical pulses delivered at very high rates, namely rates in the range above 300 Hz. This contrasts with previous approaches which typically used frequencies less than 200 Hz (indeed most studies use pulse rates at or below 130 Hz). It had previously been thought useless to use higher pulse rates because neurons can only maintain spiking-rates greater than 300 Hz for very short periods of time, primarily due to the refractory period of the neuron.

It is possible that a population of neurons, as opposed to a single neuron, might entrain to quite high frequencies. This effect may arise because, while an individual neuron may not necessarily fire on every pulse (or phase) of the stimulus, some fraction of the population of neurons will fire. Second, high rates of stimulation may act to desynchronise a population of neurons, because a low electric charge stimulus delivered at high pulse rates may tend to cause a population of neurons to desynchronise, since they all have slightly different thresholds. Irrespective of the accuracy of these hypotheses, the present invention recognises that high rate stimuli can cause “functional ablation” of the stimulated neural tissue such that it no longer functions as part of the network of activity in the brain, interrupting the epileptiform activity and terminating the seizure.

The fifth and sixth aspects of the present invention further recognise that seizures have been terminated by a sequence of electrical pulses whose pulse duration is greater than 300 μsec; for example in the range of 300-1500 μsec or 300-1000 μsec. There is a relationship between the upper limit of the pulse width and the lower limit of the rate of stimulation. For example, if the pulse width of biphasic stimulation is 1 ms per phase then the upper limit of the stimulus frequency will be 500 Hz. Associated with such long pulse durations is a very low current intensity and it is noted that seizures have been terminated with a current of only 3 μA using such stimulus pulses. The charge per phase of stimulus was 3nC/phase. The particular combination of long pulse width (1 ms) and high frequency (500 Hz) results in a waveform where there is no ‘OFF’ period in between negative and positive phase of consecutive stimuli. Thus, at a given current intensity, the rate of termination of seizures may be increased by increasing the pulse width.

The third to sixth aspects of the invention further recognise that seizures have been terminated by a sequence of electrical pulses whose pulses are aperiodic/asynchronous, e.g., with a Poisson distribution of inter-pulse intervals. This type of stimulation ensures that the timing of the pulses is not locked to any particular frequency. This ensures that rather than stimulating in a synchronous, rhythmically repeating fashion, each electrode delivers a unique series of pulses having inter-pulse intervals that over time show a Poisson distribution, characterised by a Poisson-rate (or equivalently a Poisson-frequency) equal to the pulse rate.

By applying an electrical stimulation to the brain via the at least one electrode, the electrical stimulation being pulsatile and comprising pulses forming a pulse train, wherein the pulse train has an inconstant inter pulse interval such that the pulse rate is not constant throughout the pulse train, and also wherein the pulses having a pulse width greater than substantially 300 μsec, a significant reduction in the duration of seizures can be achieved. This can be achieved at a variety of frequencies, including frequencies lower than 300 Hz. For example, the frequency of stimulation may be as low as 100 Hz, for example. The reduction may be achieved with the electrical stimulus at a current intensity that, without the specified pulse widths and aperiodic/asynchronous pulse train, would be too low to reduce the duration of seizures to any significant degree. Achieving a reduction in the duration of seizures at a lower current intensity is advantageous, as it can reduce the physical impact of the stimulus on the patient. For example, it may prevent the patient receiving a tingling sensation, or feeling uncomfortable, upon application of the electrical stimulus.

The purpose of the device is thus to disrupt or modify organised neural activity, whether this is of normal or pathological origin. The primary application is in the abatement and elimination of epileptic seizures. It is envisaged that the primary application will be in patients who have intractable epilepsies that are known to be of focal origin on clinical or neurophysiological grounds, but where the structural abnormality responsible is in an area which is either too large or inaccessible to be treated by surgical removal, or located in eloquent brain. The primary target group then are those patients with a discrete cortical focus or foci.

However, it is envisaged that a stimulation strategy in accordance with one or more of the first to sixth aspects of the invention could also have application for a number of other conditions in which organised neural activity leads to dysfunction, including epilepsies of generalised origin. It is possible that the type of electrical stimulation described here could also have therapeutic benefit for people suffering from a number of neurological disorders such as movement disorders such as Parkinson's disease, dystonias, chronic pain, and psychiatric disorders such as depression and obsessive-compulsive disorder. Such embodiments are thus encompassed within the scope of this application, notwithstanding that the primary application considered here is the abatement and elimination of epileptic seizures.

### Example 1

### Experimental Method and Materials

Male, inbred Sprague-Dawley rats (265-560 g) have been used in a modified maximal electroshock (MES) model of epilepsy. One day prior to surgical implantation and for four days after, Baytril (1.7 ml/500 ml) was introduced to the rat's water supply to aid in infection control. Animals were first anaesthetised using a combination of Ketamine (70 mg/kg i.p.) and Xylazine (10 mg/kg i.p.) to a maximal volume of 500 μl-1 ml depending on rat body mass before being located in a stereotaxic headframe. A cruciform pattern of two incisions was made over the skull, one 20 mm in length in the anterior-posterior direction and 10 mm in length at 90 degrees to the first. Sutures were used in each of the resulting triangular skin flaps to provide access to the skull. Mechanical scraping and hydrogen peroxide (3%) were used to remove connective tissue from the skull and dry the skull from any fluid. A craniotomy was created over the right motor cortex (M1) where the 16-channel microwire array (Tucker-Davis Technologies, Fl, USA) was placed. The array consisted of two parallel rows, each having eight 50 μm tungsten electrodes, one row with length 2.5 mm and the other 3 mm. Spacing between rows was 500 μm and between electrodes of the same row was 250 μm. The electrode was aligned anteroposteriorally, with its centre being level with Bregma. With the headmount secured with dental cement, the skin flaps were pulled together as much as possible and sutured together. The animal was then removed from the stereotaxic frame and placed on a heating pad for swifter recovery.

Following the above procedure it was possible to begin examining the modulating effects of cortical stimulation on clinical and electrophysiological behaviour. As purchased, the Tucker-Davis Technologies (TDT) hardware was capable of either recording or stimulating through the 16 available channels but not doing both simultaneously. Some custom modifications were made to make it possible to re-route half of the channels to the signal preamplifiers and the other half to the optically isolated stimulator. In this way it became possible to deliver a cortical stimulus and monitor its effect on neighbouring populations of neurons within the cortex.

To prevent the build up of charge at the electrode-tissue interface, which causes neuronal death, only charge balanced stimulus (biphasic) stimuli were used. Also relating to safety, McCreery, et al, (1994) states that the maximum safe amount of charge per phase (CpP for microelectrode stimulation of the cochlear nucleus is 3nC/phase, where:

CpP=I×W_{P} (W_{P} is the phase width)

The MES stimulation (50 Hz, 300 μs PW, 50 μA, 2 s duration) was delivered to the minimum number of electrodes required to elicit reliable seizures. Usually this was a single pair of stimulating/reference electrodes but occasionally all four pairs were necessary. The therapeutic stimulus then followed the MES stimulus by a temporal gap chosen by the experimenter. This window allowed the user to determine whether epileptiform afterdischarges (EADs) had successfully been initiated and thus if the therapeutic stimulus had any effect on their propagation and/or persistence. Parameters for the therapeutic stimulation primarily came from within the parameter space determined to be well tolerated from previous investigations and secondarily from literature reporting positive findings in similar animal and human studies. These parameters were also preferentially chosen to be within the 3nC/phase limit that ensures safety for underlying neural tissue. Where possible, therapeutic stimulations were not delivered through the same electrodes used in the MES stimulus, except where all four pairs of electrodes were required to initiate EADs and this was unavoidable. An abrupt cessation of EADs was taken as the gold standard, but a significant reduction in seizure duration and/or severity would also represent a positive outcome. Custom MATLAB code was written to display the EAD duration and stimulation strategies and timings of consecutive trials.

All data were acquired using the TDT hardware at a sampling frequency of 3051 Hz. All stimulus isolators were previously calibrated to ensure no DC offset was present in the delivered stimuli, further ensuring the charge balance requirements for safe neural stimulation. Determination of EAD duration was performed offline using purpose written MATLAB analysis programs. EAD duration times were also independently verified by an experienced observer of clinical EEG.

### Example 1

### Methods: Parameter Search Space

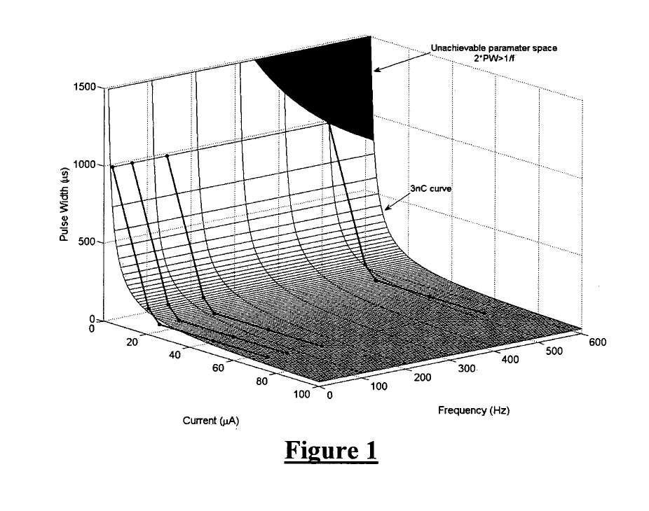

The parameter search space was designed with the a priori knowledge that the safe limit for chronic electrical stimulation to neural tissue via micro electrodes is 3nC/phase. The parameters investigated for seizure abatement or termination were the current, pulse width and frequency. Having three free variables gave a large parameter space. In order to search this space for an effective stimulation strategy in a systematic manner the strategy shown in FIG. 1 was employed. The points marked by the circles show the parameters which were investigated. The power of each parameter combination is proportional to current²×pulse width×frequency.

The upper limit for safe stimulation is drawn as a two dimensional curved surface (as depicted by the surface marked as ‘3 nC curve’ in FIG. 1). The area relating the upper limits of the frequency to the pulse width was marked on the surface as the dark shaded area (marked as ‘2×PW>1/f’ in FIG. 1). The current was set to be restricted to points lying on, or close to, the surface of the 3nC curve. The pulse width, frequency and current were varied between points marked by the circles.

It was felt that by separating the points evenly along the frequency and pulse width axes the search space could be traversed in an effective manner. By following this exploratory method the beneficial stimulation strategies of the present invention were discovered.

### Example 1

### Results: Stimulation Strategy

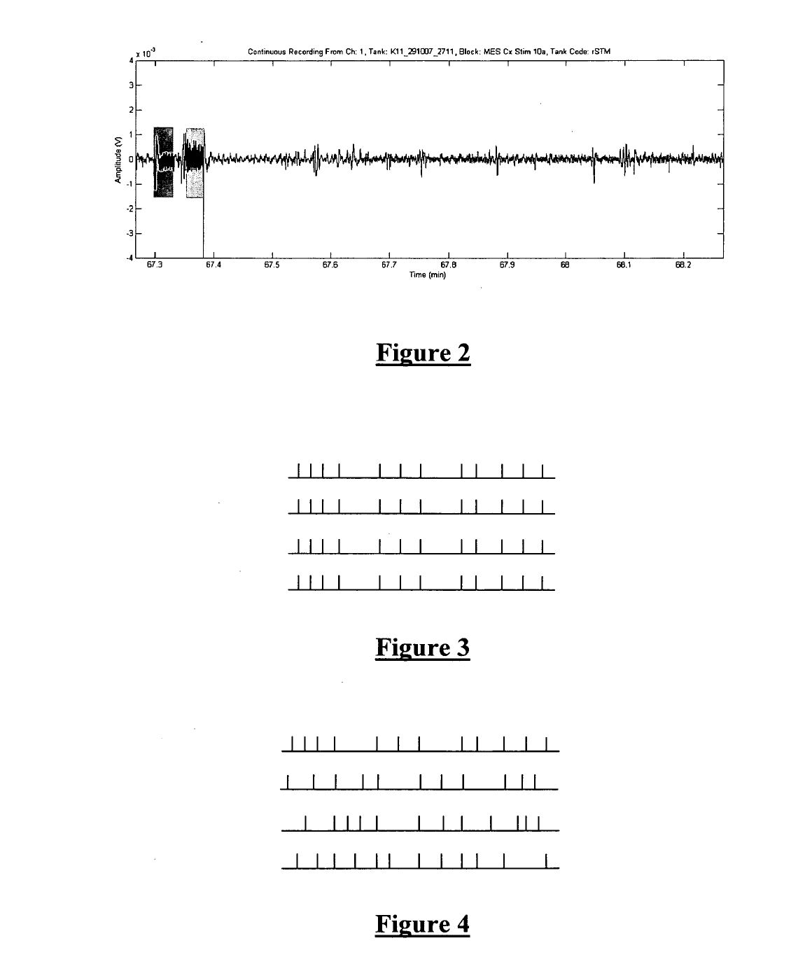

Using the experimental procedure as described above, the following stimulation strategy was developed. This strategy uses biphasic square wave pulse trains to best alleviate any potential of neural damage arising from chronic neural electrical stimulation from metal electrodes. An example of the stimulation strategy successfully abating a seizure can be seen in FIG. 2.

The parameters describing the stimulation strategy are as follows. Pulse width: the pulse width which has successfully abated seizure events has a duration of 1 ms. The parameter was discovered from our experimental procedure, as described above. Frequency: High frequency electrical stimulation (>100 Hz) is thought to have an effect of creating a functional lesion in neural tissue. There are two hypothetical mechanisms for this effect: 1) The stimulation prevents the affected neurons from transmitting useful information, which means that they no longer contribute functionally to neural circuits that they are usually a part of. 2) The stimulation generates high levels of neural activity, leading to neurotransmitters becoming depleted in the pre-synaptic cells and thereby blocking communication (and possibly also the spread of epileptic activity).

Neurons can maintain firing rates of >300 Hz for only brief periods of time. The frequency limit provides a guide as to what frequencies would be required to interrupt bursting neurons that generate epileptic activity, via the mechanisms described above. Therefore the lower frequency limit for the therapeutic stimulation frequency is around 300 Hz. The upper limit will be constrained by the pulse-width. For example, if the pulse width of biphasic stimulation is 1 ms then the upper limit of the stimulus frequency will be 500 Hz, i.e. fₘₐₓ=1/(2×PW).

Given a pulse width of 1 ms we have a possible range of 300-500 Hz for the stimulation frequency. At frequencies of <500 Hz it could be desirable to introduce some ‘jitter’ in the frequency to decrease the probability of the stimulus timing occurring within the refractory period of the neurons entrained to the epileptic activity. This is achieved in the stimulation protocol by allowing the frequency to vary around the characteristic frequency, with the inter-pulse intervals following a Poisson distribution. This distribution was chosen from the stochastic character of normal neural firing.

Another important result of using high frequency stimulation is, the highly localised nature of the receptive neural field, when delivered in a bipolar montage using adjacent electrodes. Dendritic trees of neurons in the cortex act as a filter to high frequency stimuli and the energy requirements of moving ions at high rates attenuates the stimulus over relatively short distances. Utilising this fact, stimulation could be delivered to an epileptic focus, leaving surrounding tissue minimally affected.

Stimulus pattern: The stimulation pattern is synchronously delivered to the selected electrodes. The frequency may or may not be periodic (as described above). An example stimulation trigger signal for the synchronous paradigm can be seen below in FIG. 3. Any number of electrodes may be stimulated which can be influenced by the specific pathology of the patient. The stimulations are delivered in a bipolar configuration. Stimulation around the electrodes may have two distinct paradigms, synchronous and asynchronous delivery modes. These modes refer to the specific electrodes to where the stimulus is being delivered. As opposed to the synchronous mode described above, the asynchronous mode allows each active channel to have a unique frequency and/or to follow a unique Poisson distribution. An example stimulation trigger signal for the asynchronous paradigm can be seen in FIG. 4. The asynchronous mode may be extended to incorporate variation of the reference electrode. This will add a spatial component to the random temporal delivery.

### Example 1

### Statistic Significance of Seizure Abatement

The results here focus on one measured parameter known as epileptiform afterdischarge (EAD) duration. Essentially this is the duration, in seconds, of the abnormal electrical signal arising from the brain that is measured through any of the eight recording electrodes. The varying analyses revolve around differences that exist in the distributions of these durations in the stimulated versus non-stimulated cases. Of particular interest here are the observations that begin on the 27th day post surgery and carry through to the 70th post-surgical day. Throughout the period of time included in this analysis, a range of potentially therapeutic combinations were administered in a pseudo-randomised fashion in order to avoid the confounding effects of repeated sequential delivery of the same experimental variables.

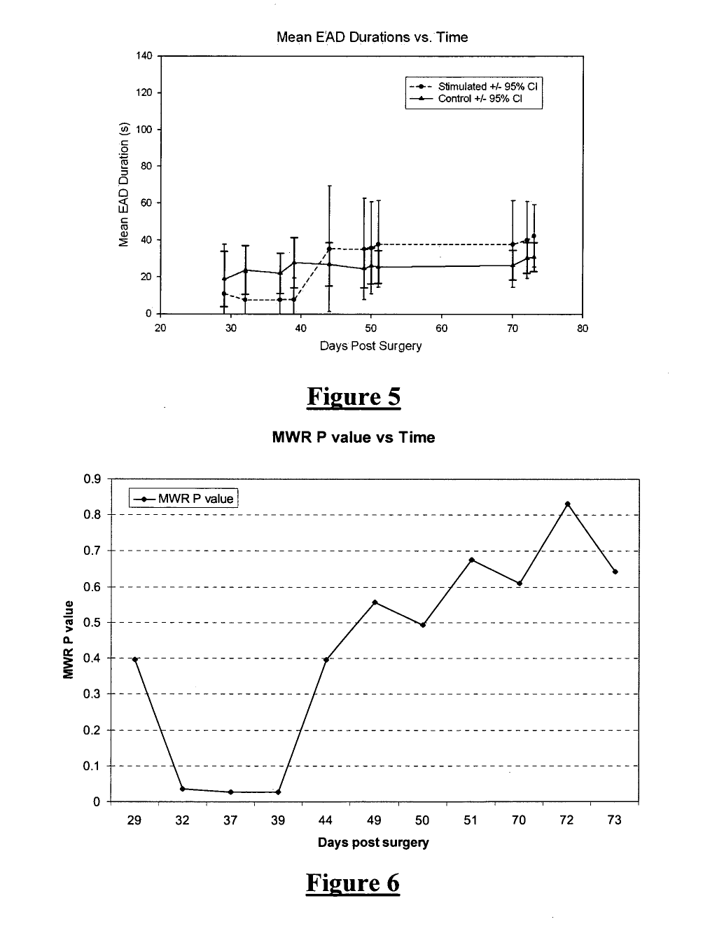

By reducing the raw data set to cumulative mean EAD durations, standard deviations and 95% confidence intervals, it was possible to use statistical analysis to identify any potentially significant differences in their distributions. This was achieved here by implementing a Mann-Whitney Rank (MWR) Sum test in the statistics analysis package SigmaStat. Results can be seen in FIGS. 5 and 6. The results show a statistically significant result (P<0.05) for days 32 to 39, after which the model deteriorates following degradation of the electrode-tissue interfaces.

### Example 2

### Experimental Methods and Materials

Female Wistar rats (250-400 g) displaying spontaneous primarily generalised seizures were used as a second model in which therapeutic stimulation strategies were tested. The pre-surgical, anaesthetic and post-surgical routines were very similar with the primary exception being the electrodes used were not microwire arrays. In this application, pairs of cortical screw electrodes or a monopolar wire-cortical screw pairing were implanted bilaterally to be aligned with M1/S1HL in the anteroposterior axis. The electrodes were placed approximately 1.5 mm anterior and 3 mm posterior to Bregma.

All data were acquired using the TDT hardware at a sampling frequency of 3051 Hz. Stimulus parameters were selected from previous studies as those combinations shown to have been previously effective in other rat models. Stimuli were typically delivered with a latency of 0.5 s to 2 s from seizure onset with control seizures left unstimulated in a pseudo-random fashion. All delivered stimuli were between 0.2 s and 0.5 s in duration. Current intensities varied between 50 μA and 650 μA, depending on the electrode geometry used. Determination of EAD duration was performed offline using purpose written MATLAB analysis programs. EAD duration times were also independently verified by an experienced observer of clinical EEG.

### Example 2

### Results: Stimulation Strategy

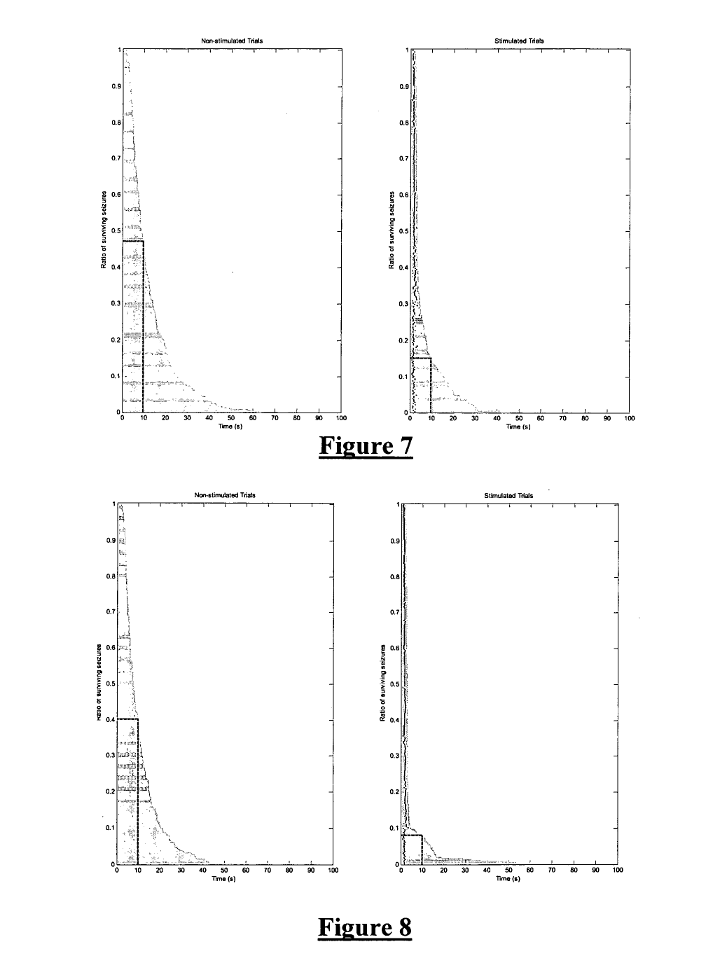

FIG. 7 shows the duration and treatment latency for the delivered stimuli and the total EAD duration for all trials of aperiodic/asynchronous, 500 Hz (average rate approximately 370 Hz), 1000 μs stimuli. Each horizontal line represents a single trial with the position of the bar in the right hand chart indicating when each stimulus was delivered in relation to the seizure start. The left hand axes contain the non-stimulated trials EAD data and the axes of the right those from all stimulated trials. FIG. 8 shows the same comparison but in this case the stimulus was set to 1000 Hz (average rate approximately 740 Hz), 500 μs, aperiodic/asynchronous. In both figures the dashed lines show the percentage of trials in which the EAD has stopped by the 10 second mark. For the non-stimulated trials in both figures, between 50-60% of all seizures had ended by 10 seconds. For the 500 Hz stimulated trials, this proportion had increased to approximately 85% of all seizures and for the 1000 Hz stimulations greater than 90% of all seizures were of 10 seconds duration or less. Together with the statistical significance presented below, these results clearly indicate a marked reduction in seizure duration when stimulation of this nature was applied.

For the 500 Hz stimulated trials, consistent with the fact that the stimuli were aperiodic/asynchronous, and the pulse width was 1000 μs, it should be noted that 500 Hz was the maximum frequency of the electrical stimuli. The average frequency across the entire stimuli in this instance was approximately 370 Hz. Similarly, for the 1000 Hz stimulated trials, consistent with the fact that the stimuli were aperiodic/asynchronous, and the pulse width was 500 μs, 1000 Hz was also the maximum frequency of the electrical stimuli. The average frequency across the entire stimuli in this instance was approximately 740 Hz.

### Example 2

### Statistical Significance of Seizure Abatement

The Mann-Whitney Rank Sum Test was again used to evaluate any statistical significance between the EAD populations of non-stimulated versus stimulated trials. For both 500 Hz and 1000 Hz stimulation significance (P<0.001) was shown.

### Example 3

### Experimental Methods and Materials; Results and Stimulation Strategy

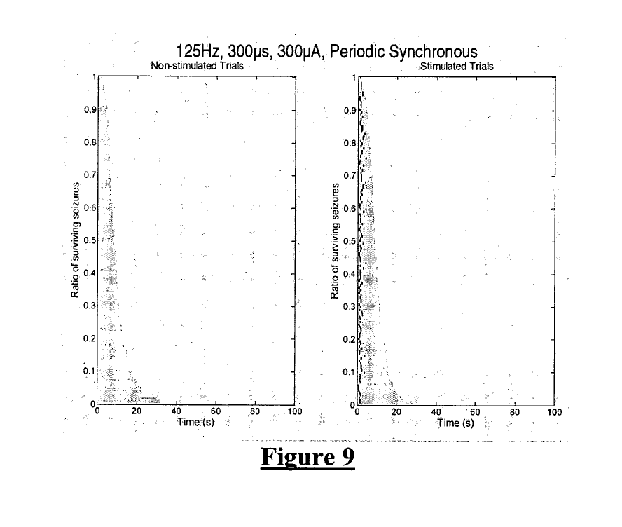

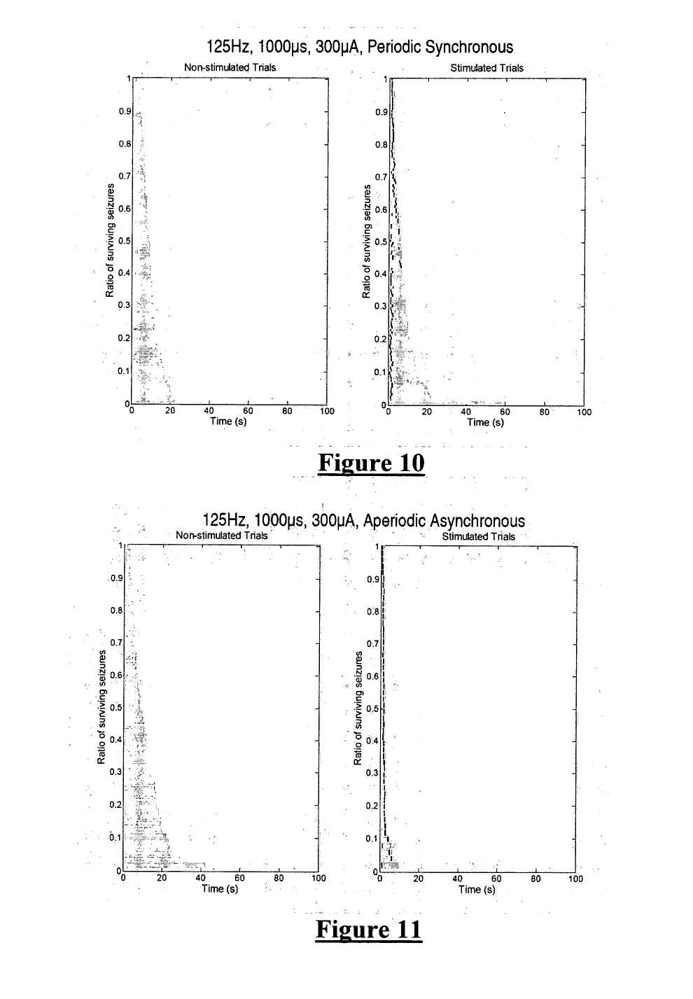

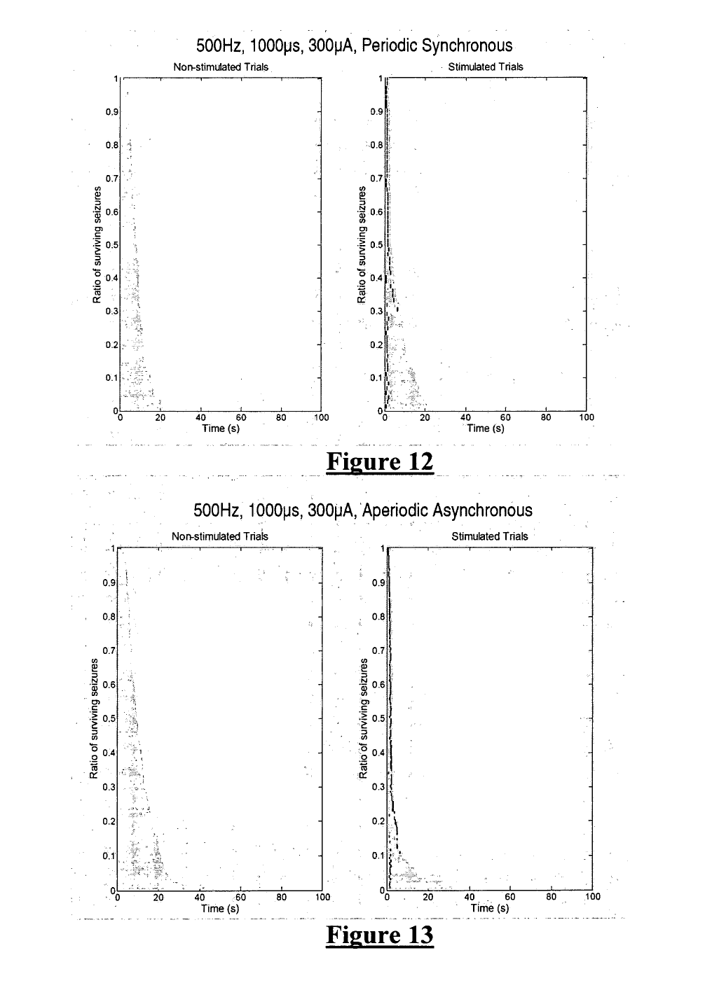

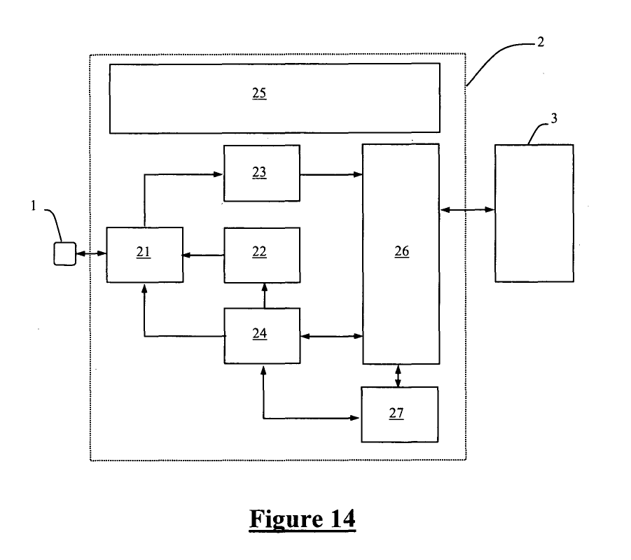

The testing approach taken with respect to Example 2 was repeated, but, with reference to the plots of FIGS. 10 to 13, testing was performed by delivery of both aperiodic/asynchronous (Ap/As) and periodic/synchronous (P/S) stimuli, at both 125 Hz and 500 Hz, and with pulse width of 1000 μs, the stimulation current being maintained at 300 μA. For the purpose of comparison, with reference to the plot of FIG. 9, testing was also performed upon delivery of periodic/synchronous stimuli, at 125 Hz, with a pulse width of 300 μs, and with the stimulation current maintained at 300 μA.

For the 500 Hz stimulated trials, where the stimuli were aperiodic/asynchronous, since the width was 1000 μs, it should be noted that 500 Hz was the maximum frequency of the electrical stimuli. The average frequency across the entire stimuli in this instance was approximately 370 Hz.

The mean seizure duration and median seizure duration were calculated for all of the seizures shown in each plot, for non stimulated (NS) trials and stimulated (S) trials, and the details are set forth in Table 1.

TABLE 1

| | | | | Mean | | Median | |
|---|---|---|---|---|---|---|---|
| | | | | seizure | | seizure | |
| | | | | duration | | duration | |
| Frequency | Pulse width | Pattern | Current | NS | S | NS | S |
| 125 Hz | 300 μs | P/S | 300 μA | 9.5 | 9.6 | 8.3 | 8.8 |
| 125 Hz | 1000 μs | P/S | 300 μA | 9.2 | 7.6 | 8.8 | 6.0 |
| 125 Hz | 1000 μs | Ap/As | 300 μA | 11.1 | 3.0 | 8.2 | 2.7 |
| 500 Hz | 1000 μs | P/S | 300 μA | 8.5 | 6.1 | 7.8 | 3.0 |
| 500 Hz | 1000 μs | Ap/As | 300 μA | 11.5 | 4.2 | 9.5 | 2.7 |

The results indicate a marked reduction in the duration of seizures upon delivery of stimulus with increased pulse width, and even greater reduction upon delivery of aperiodic/asynchronous stimulus with increased pulse width.

### Example 3

### Statistical Significance of Seizure Abatement

The Mann-Whitney Rank Sum Test was again used to evaluate any statistical significance between the EAD populations of non-stimulated versus stimulated trials. For all but the 125 Hz, 300 μs, P/S, 300 μA stimulation, significance (P<0.001) was shown.

It will be appreciated by persons skilled in the art that numerous variations and/or modifications may be made to the invention as shown in the specific embodiments without departing from the spirit or scope of the invention as broadly described. For example different electrode configurations may be used, leading to a change in electrode surface area and impacting the allowable charge densities in therapeutic stimuli. In such embodiments a change in intensity may occur to alter the dosage, while retaining functionality of the stimulus. The present embodiments are, therefore, to be considered in all respects as illustrative and not restrictive.

1. A device for counteracting seizure events in a mammalian brain, the device comprising:
at least one electrode configured to apply an electrical stimulus to the brain; and
a stimulus generator which is arranged to apply an electrical stimulation to the brain via the at least one electrode, the electrical stimulation being pulsatile and comprising pulses forming a pulse train, wherein the pulse train has an inconstant inter pulse interval such that the pulse rate is not constant throughout the pulse train, and wherein the pulses having a pulse width greater than substantially 300 μsec.

2. The device of claim 1, wherein the pulse rate is pseudo-randomly varied about the average pulse rate.

3. The device of claim 2, wherein the inter-pulse intervals vary according to a Poisson distribution.

4. (canceled)

5. The device of claim 1, wherein the pulses have a pulse width between 300 and 1200 μsec per phase.

6. The device of claim 1, wherein the pulses have a pulse width between 300 and 1000 μsec per phase.

7. (canceled)

8. The device of claim 1, wherein the average pulse rate is greater than 300 Hz.

9.-10. (canceled)

11. The device of claim 1, wherein the duty cycle of the pulse train is greater than 20%.

12. (canceled)

13. The device of claim 1, wherein the stimulus generator is configured to apply the electrical stimulation to the brain for between 300 ms and 3000 ms.

14. The device of claim 1, comprising a monitor to monitor electrical activity of the brain to detect a seizure event, wherein the stimulus generator is configured to apply the electrical stimulus only in response to a seizure event being detected.

15. The device of claim 14, wherein the monitor comprises a signal processor and a sensor and the at least one electrode serves as the sensor.

16. The device of claim 14, wherein the stimulus is initiated less than two seconds after detection of a seizure event.

17. The device of claim 14, wherein the stimulus generator is configured to adapt the parameters of the electrical stimulation in response to measured brain electrical activity before, during or after application of one or more preceding stimuli.

18. The device of claim 1 comprising:
an implanatable component, the implantable component comprising the electrode(s) and an internal antenna; and
an external component comprising a power source, the processor, and an external antenna for wireless transcutaneous communication with the internal antenna.

19. The device of claim 18, wherein the internal antenna and external antenna comprise inductive coils for effecting inductive transcutaneous power transmission to power the implantable component.

20. A device for counteracting seizure events in a mammalian brain, the device comprising:
at least one electrode configured to apply an electrical stimulus to the brain; and
a stimulus generator which is arranged to apply an electrical stimulation to the brain via the at least one electrode, the electrical stimulation being pulsatile and comprising pulses forming a pulse train having a frequency greater than substantially 300 Hz and at a duty cycle greater than substantially 20%.

21. The device of claim 20, wherein the pulse train has an inconstant inter pulse interval such that the pulse rate is not constant throughout the pulse train.

22. The device of claim 21, wherein the pulse rate is pseudo-randomly varied about the average frequency, the average frequency constituting said electrical stimulation frequency.

23. The device of claim 22, wherein the variation in inter-pulse intervals is as per a Poisson distribution.

24. The device of claim 20, wherein the frequency is greater than 350 Hz.

25.-31. (canceled)

32. The device of claim 20, wherein the duty cycle of the pulse train is greater than 30%.

33. The device of claim 20, wherein the duty cycle of the pulse train is greater than 50%.

34.-35. (canceled)

36. A method for counteracting seizure events in a mammalian brain, the method comprising applying an electrical stimulus to the brain, the electrical stimulus being pulsatile and comprising pulses forming a pulse train, wherein the pulse train has an inconstant inter pulse interval such that the pulse rate is not constant throughout the pulse train, and wherein the pulses having a pulse width greater than substantially 300 μsec.

37. The method of claim 36, wherein the pulse train has an inconstant inter pulse interval such that the pulse rate is not constant throughout the pulse train.

38. The method of claim 37, wherein the pulse rate is pseudo-randomly varied about the average pulse rate.

39. The method of claim 38, wherein the variation of the inter-pulse interval is as per a Poisson distribution.

40. (canceled)

41. The method of claim 36, wherein the pulses have a pulse width between 300 and 1200 μsec per phase.

42. (canceled)

43. The method of claim 36, wherein the average pulse rate is greater than 300 Hz.

44.-45. (canceled)

46. The method of claim 36, wherein the duty cycle of the pulse train is greater than 20%.

47. (canceled)

48. The method of claim 36, wherein the stimulus generator is configured to apply the electrical stimulation to the brain for between 300 ms and 3000 ms.

49. The method of claim 36 comprising monitoring electrical activity of the brain to detect a seizure event, and applying the electrical stimulus only in response to a seizure event being detected.

50. The method of claim 49, wherein the stimulus is initiated less than two seconds after detection of a seizure event.

51. The method of claim 36, comprising adapting the parameters of the electrical stimulation in response to measured brain electrical activity before, during or after application of one or more preceding stimuli.

52. A method for counteracting seizure events in a mammalian brain, the method comprising applying an electrical stimulus to the brain, the electrical stimulus being pulsatile and comprising pulses forming a pulse train at a frequency greater than substantially 300 Hz and at a duty cycle greater than substantially 20%.

53. The method of claim 52, wherein the pulse train has an inconstant inter pulse interval such that the pulse rate is not constant throughout the pulse train.

54. The method of claim 53, wherein the pulse rate is pseudo-randomly varied about the average pulse rate, the average pulse rate constituting said frequency of stimulation.

55. The method of claim 54, wherein the inter-pulse intervals vary according to a Poissonian distribution.

56. The method of claim 52, wherein the frequency is greater than 350 Hz.

57.-63. (canceled)

64. The method of claim 52, wherein the duty cycle of the pulse train is greater than 30%.

65. The method of claim 52, wherein the duty cycle of the pulse train is greater than 50%.

66.-67. (canceled)
